(19) United States
(12) Patent Application Publication
(10) Pub. No.: US 2023/0000741 A1
(43) Pub. Date: Jan. 5, 2023

(54) SOLID COSMETIC COMPOSITION

(71) Applicant: CHANEL PARFUMS BEAUTE, NEUILLY SUR SEINE (FR)

(72) Inventors: Lina DUPUIS, PANTIN CEDEX (FR); Cyril GIBERT, PANTIN CEDEX (FR)

(73) Assignee: CHANEL PARFUMS BEAUTE, NEUILLY SUR SEINE (FR)

(21) Appl. No.: 17/624,176

(22) PCT Filed: Jul. 3, 2020
(86) PCT No.: PCT/FR2020/051177

§ 371 Date: Dec. 30, 2021

(30) Foreign Application Priority Data
Jul. 3, 2019 (FR) 19 07389

(51) Int. Cl. A61K 8/36; A61K 8/27; A61K 8/92; A61K 8/25; A61Q 19/00; A61K 8/02

(52) U.S. Cl. CPC A61K 8/36; A61K 8/27; A61K 8/92; A61K 8/25; A61Q 19/001; A61Q 19/005; A61K 8/0216

(57) ABSTRACT

A solid cosmetic composition including 12 to 50% by weight of a non-volatile liquid fatty phase and 40 to 85% by weight of a pulverulent phase including spherical fillers that are surface-treated with a metallic soap. Also, a process for preparing such a solid cosmetic composition and a method for making up the skin or lips using such a solid cosmetic composition.

This invention relates to a solid cosmetic composition comprising 12 to 50% by weight of a non-volatile liquid fatty phase, and 40 to 85% by weight of a pulverulent phase comprising spherical fillers surface-treated with a metallic soap. The invention also relates to a process for preparing such a solid cosmetic composition and to a method for making up the skin or lips which makes use of it.

### TECHNICAL FIELD

The formulations conventionally used for solid compositions are generally loose or compact powders. By way of illustration and without limitation to the solid formulations more particularly considered in the field of cosmetics, we can cite in particular powders for the complexion, blushes, or eye shadows.

The aforementioned powders primarily have the function of adding color, eliminating shine, or providing coverage. In general, powders combine a largely predominant pulverulent phase with an at least partly liquid fatty phase constituting the binder and making it possible, in the context of compact powders, to ensure good cohesion of the pulverulent phase.

A pulverulent phase is formed essentially of fillers and coloring agents, their amounts being modulated to provide the desired makeup effect: color, coverage, or shine elimination. When the percentage of pulverulent phase in the product becomes too high, its manufacture and its compaction become complicated or even impossible to carry out at an industrial scale given the quality and productivity requirements. In addition, such solid compositions with a high content of pulverulent phase can have the disadvantage of being uncomfortable, too dry, and too powdery, and for some, of being fragile, brittle, and with poor impact resistance. Lastly, large amounts of pulverulent phase in the compact powder do not provide satisfactory sensory properties, making them difficult to pick up when collecting the powder from its packaging (“pick-up”) and/or to spread when applied to the surface of the skin to be made up (“pay-off”).

To obtain a composition in solid form, it is known to implement a compaction process (“dry process”) consisting of mixing the pulverulent phase and the fatty phase and compacting the resulting composition under high pressure in a case. Alternatively, a process referred to as a “wet process” (or “slurry”) can be implemented to produce such compositions. In this type of process, the pulverulent phase and the fatty phase of said composition are exposed to a volatile solvent so as to form a suspension, which is then pressed and the volatile solvent eliminated.

Regardless of the process considered, the amount of fatty phase, and in particular of oils, generally does not exceed 10% of the composition so as to obtain good compaction of the powder via mechanical means, and also to avoid any overflow of the composition from the case. For these reasons, these formulations often require the formulators to reduce the amount of fatty phase, and in particular of oil(s), so as to ensure proper compaction of the powder.

When the amount of fatty binder is increased, shaping the solid composition by compaction therefore becomes complicated, if not impossible. In response to this industrial constraint, more pasty solid products, for example obtained by extrusion, have been proposed. However, in the presence of too high of a content of binder phase, the composition has a tendency to wax, meaning to harden during use, and become too dense to the point of preventing the collection of product and altering its spreading properties when applied.

The search continues for solid cosmetic compositions exhibiting good cohesion and good impact resistance, which in particular can be stored and transported freely by the user without crumbling or cracking, its texture allowing easy pick-up and application.

The Applicant has unexpectedly discovered that a composition exhibiting such properties, which in principle seem incompatible, could be obtained by using, in a solid composition, a specific content of a particular liquid fatty phase, and a pulverulent phase at least partly surface-treated with a metallic soap.

The object of the invention is thus, according to a first aspect, a solid cosmetic composition comprising:
12 to 50% by weight of a non-volatile liquid fatty phase, and
40 to 85% by weight of a pulverulent phase comprising spherical fillers surface-treated with a metallic soap,
   the percentages being expressed by weight, relative to the total weight of the composition.

In particular, the invention relates to a solid cosmetic composition comprising:
12 to 50% by weight of a pulverulent phase comprising spherical fillers surface-treated with a metallic soap and further comprising a lamellar filler surface-treated with a metallic soap,
   the percentages being expressed by weight, relative to the total weight of the composition.

In particular, the invention relates to a solid cosmetic composition comprising:
13 to 35% by weight of non-volatile liquid fatty phase, and
50% to 80% by weight, preferably 60 to 75% by weight, of a pulverulent phase comprising spherical and/or lamellar fillers surface-treated with a metallic soap, the percentages being expressed by weight, relative to the total weight of the composition.

Another object of the invention, according to a second aspect, is a process for preparing such a composition, comprising:
premixing the powders constituting the pulverulent phase
preparing a fatty binder comprising the non-volatile liquid fatty phase
mashing the powders in with the fatty binder by extrusion, and
shaping the composition by pressing.

Another object of the invention, according to a third aspect, is a method for making up the skin or lips, consisting of applying such a solid cosmetic composition to the skin or lips.

Formulation

The composition according to the invention is solid, in the sense that it does not flow under its own weight. It is preferably in the form of a pasty product, obtained by extruding a mixture of a pulverulent phase and a fatty phase, optionally in the presence of a volatile solvent which will be evaporated (“slurry” process).

In particular, the composition according to the invention has a hardness of between 30 g and 250 g, preferably between 50 g and 230 g, more preferably between 80 g and 200 g.

The hardness of the composition, which is expressed in grams (g), is determined by measuring the compressive force measured at 20° C. using a texture analyzer sold under the name “TA.XT Plus Microstable System” by the Swantech company. The texture analyzer is equipped with a Stable Micro System cylinder probe ref P/2 2 mm in diameter made of stainless steel moving at the measurement speed of 1 mm/s and penetrating the composition to a depth of 3 mm with a force of 3 mm. The hardness value is the measured compressive force divided by the surface area of the texture analyzer's cylinder probe in contact with the composition. The measurement is made in 15 ml perfume jars 40 mm in height and 38 mm in diameter, filled up to the neck with the composition at room temperature, i.e. to 20 mm in height. The samples thus prepared are stored for 24 h at 20° C. before performing the measurement.

Fatty Phase

The composition according to the invention comprises at least one non-volatile liquid fatty phase, meaning a fatty phase comprising at least one non-volatile oil.

“Non-volatile oil” is understood to mean an oil which remains on keratin fibers at room temperature and atmospheric pressure for at least several hours and in particular having a vapor pressure of less than 10⁻³ mmHg (0.13 Pa).

The non-volatile oils may, in particular, be selected from hydrocarbonated and fluorinated oils and/or non-volatile siliconated oils.

For a non-volatile hydrocarbonated oil, mention may in particular be made of:
hydrocarbonated oils of animal origin,
hydrocarbonated oils of plant origin such as C4 to C36 linear alkanes, preferably C11-C21, such as phyto squalane or Emogreen L15 from SEPPIC (C15-19 alkane), or even such as phytostearyl esters, such as phytosteryl oleate, physosteryl isostearate, and phytosteryl/octyldodecyl/lauroyl glutamate (AJINOMOTO, ELDEW PS203), triglycerides composed of esters of fatty acids and of glycerol, in particular, in which the fatty acids can have chain lengths varying from C4 to C36, and in particular from C18 to C36; these oils may be linear or branched, saturated or unsaturated; these oils may, in particular, be heptanoic or octanoic triglycerides, shea oil, alfalfa oil, poppy oil, ambercup oil, millet oil, barley oil, quinoa oil, rye oil, candlenut oil, passionflower oil, shea butter, aloe vera oil, sweet almond oil, peach kernel oil, peanut oil, argan oil, avocado oil, baobab oil, borage oil, broccoli oil, calendula oil, camelina oil, carrot oil, safflower oil, hemp oil, rapeseed oil, cottonseed oil, coconut oil, pumpkin seed oil, wheat germ oil, jojoba oil, lily oil, macadamia oil, corn oil, meadowfoam seed oil, St. John's wort oil, monoi oil, hazelnut oil, apricot kernel oil, walnut oil, olive oil, evening primrose oil, palm oil, blackcurrant seed oil, kiwi seed oil, grape seed oil, pistachio oil, ambercup oil, pumpkin oil, quinoa oil, musk rose oil, sesame oil, soybean oil, sunflower oil (Helianthus Annuus seed oil), castor oil, and watermelon oil, ethyl olivate such as Vegeflow D10 from the Innovation Company, and mixtures thereof, or caprylic/capric triglycerides, such as those sold by the STEARINERIE DUBOIS company or those sold under the names MIGLYOL 810®, 812® and 818® by the DYNAMIT NOBEL company,
synthetic ethers having 10 to 40 carbon atoms;
synthetic esters, such as oils of formula R1-COOR2, in which R1 represents a residue of a linear or branched fatty acid comprising from 1 to 40 carbon atoms and R2 represents a hydrocarbon chain, in particular a branched chain containing from 1 to 40 carbon atoms provided that R1+R2 is ≥10. The esters can be, in particular, selected from esters of alcohol and of fatty acids, for example such as cetostearyl octanoate, esters of isopropyl alcohol, such as isopropyl myristate, isopropyl palmitate, ethyl palmitate, 2-ethyl-hexyl palmitate, isopropyl stearate or isostearate, isostearyl isostearate, octyl stearate, hydroxylated esters, such as isostearyl lactacte, octyl hydroxystearate, diisopropyl adipate, heptanoates, and in particular isostearyl heptanoate, octanoates, decanoates, or ricinoleates of alcohols or polyalcohols, such as propylene glycol dioctanoate, cetyl octanoate, tridecyl octanoate, 4-diheptanoate, and 2-ethyl hexyl palmitate, alkyl benzoate, polyethylene glycol diheptanoate, propylene glycol diethyl 2-hexanoate, and mixtures thereof, C12-C15 alcohol benzoates, hexyl laurate, neopentanoic acid esters, such as isodecyl neopentanoate, isotridecyl neopentanoate, isostearyl neopentanoate, octyldocecyl neopentanoate, isononanoic acid esters, such as isononyl isononanoate, isotridecyl isononanoate, octyl isononanoate, hydroxyl esters such as isostearyl lactate, diisostearyl malate;
esters of polyols and esters of pentaerythritol, such as dipentaerythritol tetrahydroxystearate/tetraisostearate,
esters of diol dimers and diacid dimers, such as Lusplan DD-DA5® and Lusplan DD-DA7®, sold by the NIPPON FINE CHEMICAL company and described in application US 2004-175338,
copolymers of diol dimers and diacid dimers and their esters, such as the copolymers dimer dilinoleyl/dimer dilinoleate and their esters, such as, for example, Plandool-G,
copolymers of polyols and of diacid dimers, and their esters, such as Hailuscent ISDA,
fatty alcohols that are liquid at room temperature with a branched and/or unsaturated carbon chain having 12 to 26 carbon atoms, such as 2-octyldodecanol, isostearyl alcohol, oleic alcohol, 2-hexyldecanol, 2-blatyloctanol, and 2-undecylpentadecan-1-ol,
C₁₂-C₂₂ higher fatty acids, such as oleic acid, linoleic acid, and mixtures thereof,
dialkyl carbonates, the 2 alkyl chains being identical or different, such as dicaprylyl carbonate sold by COGNIS under the name CETIOL CC®,
oils of high molar mass having, in particular, a molar mass ranging from approximately 400 to approximately 10,000 g/mol, in particular from approximately 650 to approximately 10,000 g/mol, in particular from approximately 750 to approximately 7500 g/mol, and more particularly varying from approximately 1000 to approximately 5000 g/mol,
siliconated oils, such as phenyl silicones for example BELSIL PDM 1000 from the WACIER company (MM=9000 g/mol) or non-phenylated silicone oils such as non-volatile polydimethylsiloxanes (PDMS), PDMS comprising alkyl or alkoxy pendant groups and/or groups at the end of the silicone chain, groups each having from 2 to 24 carbon atoms, phenylated silicones, such as phenyl trimethicones, phenyl dimethicones, phenol trimethylsiloxy diphenylsiloxanes, diphenyl dimethicones, diphenyl methyldiphenyl trisiloxanes, and 2-phenylethyl trimethylsiloxysilicates, dimethicones or phenyltrimethicone with a viscosity of less than or equal to 100 cSt, and mixtures thereof,
the fluorinated oils which can be used in the invention are in particular fluorosiliconated oils, fluorinated polyethers, fluorinated silicones as described in document EP-A-847752.

According to one particular embodiment, the non-volatile liquid fatty phase comprises at least one non-volatile oil selected from hydrocarbonated oils, non-phenylated siliconated oils, and mixtures thereof.

According to another more particular embodiment, the non-volatile oil is selected from hydrocarbonated oils, and the composition comprises from 30% to 50% by weight, relative to the total weight of the composition, of nacres surface-treated by metallic soap. Alternatively, the non-volatile oil is selected from non-phenylated siliconated oils.

The composition according to the invention may also comprise a volatile liquid fatty phase, i.e. a fatty phase comprising at least one volatile oil.

For the purposes of the invention, the term “volatile oil” is understood to mean an oil capable of evaporating in less than an hour upon contact with keratin fibers, at room temperature and atmospheric pressure. The volatile organic solvent(s) and the volatile oils of the invention are volatile cosmetic oils and organic solvents that are liquid at room temperature, having a non-zero vapor pressure, at room temperature and atmospheric pressure, ranging in particular from 0.13 Pa to 40,000 Pa (10⁻³ to 300 mmHg), in particular ranging from 1.3 Pa to 13,000 Pa (0.01 to 100 mmHg), and more particularly ranging from 1.3 Pa to 1300 Pa (0.01 to 10 mmHg).

In particular, the volatile liquid fatty phase can comprise at least one volatile oil selected from hydrocarbonated oils, siliconated oils, and mixtures thereof.

The volatile oil can be hydrocarbonated. The hydrocarbonated volatile oil can be selected from hydrocarbonated oils having from 7 to 16 carbon atoms. For a hydrocarbonated volatile oil having from 7 to 16 carbon atoms, we can cite in particular branched C8-C16 alkanes such as C8-C16 iso-alkanes (also called isoparaffins), isododecane, isodecane, isohexadecane, and for example the oils sold under the trade names of Isopar or Permyl, branched C8-C16 esters such as isohexyl neopentanoate, and mixtures thereof. Preferably, the volatile hydrocarbonated oil having from 8 to 16 carbon atoms is selected from isododecane, isodecane, isohexadecane, and mixtures thereof, and in particular is isododecane.

The volatile oil can be a volatile linear alkane. According to one embodiment, an alkane suitable for the invention can be a volatile linear alkane comprising from 7 to 14 carbon atoms. Such a volatile linear alkane can advantageously be of plant origin. As an example of alkanes suitable for the invention, we can cite the alkanes described in patent applications WO 2007/1068371 or WO2008/155059 of the Cognis company (mixtures of distinct alkanes differing by at least one carbon). These alkanes are obtained from fatty alcohols, which themselves are obtained from coconut or palm oil. As examples of linear alkanes suitable for the invention, we can cite n-heptane (C7), n-octane (C8), n-nonane (C9), n-decane (010), n-undecane (C11), n-dodecane (C12), n-tridecane (C13), n-tetradecane (C14), and mixtures thereof. According to one particular embodiment, the volatile linear alkane is selected from n-nonane, n-undecane, n-dodecane, n-tridecane, n-tetradecane, and mixtures thereof. According to a preferred embodiment, we can cite the mixtures of n-undecane (C11) and n-tridecane (C13) obtained in examples 1 and 2 of application WO2008/15505 of the Cognis company. We can also cite the mixture of n-undecane (C11) and n-tridecane (C13) sold by the BASF company under the name CETIOL ULTIMATE. We can also cite n-dodecane (C12) and n-tetradecane (C14) sold by Sasol respectively under the names PARAFOL 12-97 and PARAFOL 14-97, as well as their mixtures. We can also cite the C9-C12 alkane marketed under the name VEGELIGHT SILK by the Biosynthis company. It is possible to use the volatile linear alkane alone or preferably a mixture of at least two distinct volatile linear alkanes, differing from each other by a carbon number n of at least 1, in particular differing from each other by a carbon number of 1 or 2.

The volatile oil can be a volatile siliconated oil such as cyclic polysiloxanes, linear polysiloxanes, and mixtures thereof. As linear volatile polysiloxanes, we can cite hexamethyldisiloxane, octamethyltrisiloxane, decamethyltetrasiloxane, tetradecamethylhexasiloxane, and hexadecamethylheptasiloxane. As volatile cyclic polysiloxanes, we can cite hexamethylcyclotrisiloxane, octamethylcylotetrasiloxane, decamethylcyclopentasiloxane, and dodecamethylcyclohexasiloxane.

Additionally or alternatively, the composition produced may comprise at least one fluorinated volatile oil.

According to a preferred embodiment, the composition according to the invention is without any volatile liquid fatty phase, i.e. without any volatile oil. The term “volatile oil” is understood to mean a composition comprising less than 3% by weight of volatile oil, preferably less than 1% by weight, and more preferably comprising no volatile oil.

The composition according to the invention may also comprise a solid fatty phase, comprising at least one wax and/or a pasty fatty substance and/or a lipophilic gelling agent.

According to a preferred embodiment, the composition according to the invention comprises from 0.1 to 15% by weight, preferably from 0.5 to 10% by weight, of a solid fatty phase.

### Waxes

The composition according to the invention may comprise at least one wax.

The wax considered in the context of the invention is generally a lipophilic compound, solid at room temperature (25° C.), having a reversible solid/liquid state change, having a melting point greater than or equal to 30° C. and which may be as high as 120° C.

In particular, the waxes suitable for the invention can have a melting point greater than approximately 45° C., and in particular greater than 55° C. The melting point of the wax can be measured using a differential scanning calorimeter (D.S.C.), for example the calorimeter sold under the name DSC 30 by the METLER company.

The waxes capable of being used in the compositions of the invention are selected from solid waxes which are deformable or not deformable at room temperature, of animal, plant, mineral, or synthetic origin, and mixtures thereof.

The wax may also have a hardness ranging from 0.05 MPa to 30 MPa, and preferably ranging from 6 MPa to 15 MPa. The hardness is determined by measuring the compressive force measured at 20° C. using the texture analyzer sold under the name TA-TX2i by the RHEO company, equipped with a stainless steel cylinder having a diameter of 2 mm and moving at the measurement speed of 0.1 mm/s, and penetrating into the wax to a penetration depth of 0.3 mm.

It is possible in particular to use hydrocarbon waxes such as lanolin wax, and Chinese insect waxes; rice wax, Carnauba wax, Candellila wax, Ouricury wax, esparto wax, cork fiber wax, sugar cane wax, Japanese wax, and sumac wax; montan wax, microcrystalline waxes, paraffins and ozokerite; beeswax, jojoba wax, mimosa wax, sunflower wax, polyethylene waxes, waxes obtained by Fisher-Tropsch synthesis, and waxy copolymers as well as their esters. A mixture of jojoba wax, mimosa wax, sunflower wax is for example marketed under the name ACTICIRE MP by the GATTEFOSSE company. In particular, the hydrocarbon waxes can be selected from Carnauba wax, beeswax, jojoba wax, mimosa wax, sunflower wax, and mixtures thereof.

We can also cite waxes obtained by catalytic hydrogenation of animal or vegetable oils having C₈-C₃₂ linear or branched fatty chains.

Among these, we can cite in particular hydrogenated jojoba oil, hydrogenated sunflower oil, hydrogenated castor oil, hydrogenated coconut oil, and hydrogenated lanolin oil, bis(1,1,1-trimethylolpropane) tetrastearate sold under the name “HEST 2T-4S” by the HETERENE company, bis(1,1,1-trimethylolpropane) tetrabehenate sold under the name HEST 2T-4B by the HETERENE company.

It is also possible to use waxes obtained by transesterification and hydrogenation of vegetable oils, such as castor or olive oil, for example the waxes sold by the SOPHIM company under the names Phytowax ricin 16L64® and 22L73® and Phytowax Olive 18L57. Such waxes are described in application FR-A-2792190.

It is also possible to use silicone waxes which can advantageously be substituted polysiloxanes, preferably with a low melting point. These silicone waxes are known or can be prepared according to known methods. Among the commercial silicone waxes of this type, we can cite in particular those sold under the names Abilwax 9800, 9801, or 9810 (GOLDSCHMIDT), KF910 and KF7002 (SHIN ETSU), or 176-1118-3 and 176-11481 (GENERAL ELECTRIC), the alkyl- or alkoxydimethicones such as the following commercial products: Abilwax 2428,2434, and 2440 (GOLDSCHMIDT), or VP 1622 and VP 1621 (WACKER), as well as (C20-C60) alkyl dimethicones, in particular (C30-C45) alkyl dimethicones such as the silicone wax sold under the name SF-1642 by the GE-Bayer Silicones company.

It is also possible to use hydrocarbon waxes modified with siliconated or fluorinated functional groups such as, for example: siliconyl candelilla, siliconyl beeswax, and Fluorobeeswax from Koster Keunen.

The waxes may also be selected from fluorine waxes.

According to one particular embodiment, the compositions according to the invention may comprise at least one wax called a sticky wax. For the sticky wax, a C20-C40 alkyl (hydroxystearyloxy)stearate (the alkyl group comprising from 20 to 40 carbon atoms) can be used, alone or in a mixture, in particular a C20-C40 alkyl 12-(12′-hydroxystearyloxy)stearate. Such a wax is sold in particular under the names “Kester Wax K 82 P®” and “Kester Wax K 80 P®” by the KOSTER KEUNEN company.

According to a preferred embodiment, the waxes are selected from hydrocarbon waxes, preferably selected from Carnauba wax, beeswax, jojoba wax, mimosa wax, sunflower wax, and mixtures thereof.

Pasty Fatty Substance

The solid fatty phase may also comprise a pasty, hydrocarbonated, siliconated, and/or fluorinated fatty substance, or a mixture thereof.

For the purposes of the invention, the term “pasty fatty substance” is understood to mean a lipophilic fatty compound with a reversible solid/liquid state change exhibiting an anisotropic crystal structure in the solid state, and comprising a liquid fraction and a solid fraction at a temperature of 23° C.

In other words, the onset melting temperature of the pasty fatty substance may be less than 23° C. The liquid fraction of the pasty fatty substance measured at 23° C. can represent 9 to 97% by weight of the pasty fatty substance. This fraction which is liquid at 23° C. preferably represents between 15 and 85%, more preferably between 40 and 85%, by weight.

For the purposes of the invention, the melting temperature corresponds to the temperature of the most endothermic peak observed in thermal analysis (DSC) as described in standard ISO 11357-3; 1999. The melting point of a pasty fatty substance can be measured using a differential scanning calorimeter (DSC), for example the calorimeter sold under the name “MDSC 2920” by the TA Instruments company.

The measurement protocol is as follows:

A sample of 5 mg of pasty fatty substance placed in a crucible is subjected to a first rise in temperature from −20° C. to 100° C., at the heating rate of 10° C./minute, then cooled from 100° C. to −20° C. at a cooling rate of 10° C./minute, and finally subjected to a second rise in temperature from −20° C. to 100° C. at a heating rate of 5° C./minute. During the second rise in temperature, the variation in the difference in power absorbed by the empty crucible and by the crucible containing the sample of pasty fatty substance is measured as a function of the temperature. The melting point of the pasty fatty substance is the value of the temperature corresponding to the top of the peak of the curve representing the variation of the difference in power absorbed as a function of the temperature.

The liquid fraction by weight of the pasty fatty substance at 23° C. is equal to the ratio of the enthalpy of fusion consumed at 23° C. to the enthalpy of fusion of the pasty fatty substance. The enthalpy of fusion of the pasty fatty substance is the enthalpy consumed by said substance in order to transition from the solid state to the liquid state. The pasty fatty substance is said to be in the solid state when all of its mass is in crystalline solid form. The pasty fatty substance is said to be in the liquid state when all of its mass is in liquid form.

The enthalpy of fusion of the pasty fatty substance is equal to the area under the curve of the thermogram obtained using a differential scanning calorimeter (DSC), such as the calorimeter sold under the name MDSC 2920 by the TA Instrument company, with a rise in temperature of 5 or 10° C. per minute, according to ISO 11357-3: 1999.

The enthalpy of fusion of the pasty fatty substance is the amount of energy required to cause the pasty fatty substance to transition from the solid state to the liquid state. It is expressed in J/g.

The enthalpy of fusion consumed at 23° C. is the amount of energy absorbed by the sample in order to transition from the solid state to the state it presents at 23° C., consisting of a liquid fraction and a solid fraction.

The liquid fraction of the pasty fatty substance measured at 32° C. preferably represents from 30 to 100% by weight of the pasty fatty substance, preferably from 50 to 100%, more preferably from 60 to 100% by weight of the pasty fatty substance. When the liquid fraction of the pasty fatty substance measured at 32° C. is equal to 100%, the temperature at the end of the melting range of the pasty fatty substance is less than or equal to 32° C.

The liquid fraction of the pasty fatty substance measured at 32° C. is equal to the ratio of the enthalpy of fusion consumed at 32° C. to the enthalpy of fusion of the pasty fatty substance. The enthalpy of fusion consumed at 32° C. is calculated in the same way as the enthalpy of fusion consumed at 23° C.

The pasty fatty substance is preferably selected from synthetic fatty substances and fatty substances of plant origin. A pasty fatty substance can be obtained by synthesis starting with products of plant origin.

The pasty fatty substance is advantageously selected from:
lanolin and its derivatives,
polyol ethers selected from pentaerythritol ethers and polyalkylene glycol ethers,
fatty alcohol and sugar ethers, and mixtures thereof, pentaerythritol ether and polyethylene glycol ether comprising 5 oxyethylene units (5 OE) (CTFA name: PEG-5 Pentaerythrityl Ether), pentaerythritol ether and polypropylene glycol ether comprising 5 oxypropylene units (5 OP) (CTFA name: PPG-5 Pentaerythrityl Ether), and mixtures thereof, and more particularly the mixture PEG-5 Pentaerythrityl Ether, PPG-5 Pentaerythrityl Ether, and soybean oil, marketed under the name “Lanolide” by the VEVY company, a mixture in which the constituents are found in a 46/46/8 weight ratio: 46% PEG-5 Pentaerythrityl Ether, 46% PPG-5 Pentaerythrityl Ether, and 8% soybean oil,
polymeric or non-polymeric silicone compounds,
polymeric or non-polymeric fluorinated compounds,
vinyl polymers, in particular olefin homopolymers and copolymers, hydrogenated diene homopolymers and copolymers,
liposoluble polyethers resulting from polyetherification between one or more C2-C100 diols, preferably C2-050,
esters,
   and/or mixtures thereof.

The pasty fatty substance is preferably a polymer, in particular a hydrocarbon polymer.

Among the liposoluble polyethers, preference is given in particular to copolymers of ethylene oxide and/or of propylene oxide with long chain C6-C30 alkylene oxides, more preferably such that the weight ratio of ethylene oxide and/or propylene oxide to alkylene oxides in the copolymer is 5:95 to 70:30. In this family, mention is made in particular of copolymers such as long-chain alkylene oxides arranged in blocks having an average molecular weight of 1,000 to 10,000, for example a block copolymer of polyoxyethylene/polydodecyl glycol such as ethers of dodecanediol (22 mol) and of polyethylene glycol (45 EO) sold under the brand ELFACOS ST9 by AKZO NOBEL.

Among the esters, the following are preferred:
esters of an oligomeric glycerol, in particular esters of diglycerol, in particular condensates of adipic acid and of glycerol, for which part of the hydroxyl groups of the glycerols have reacted with a mixture of fatty acids such as stearic acid, capric acid, stearic acid, isostearic acid, and 12-hydroxystearic acid, in particular such as those marketed under the brand Softisan649 by the SASOL company,
arachidyl propionate marketed under the brand Waxenol 801 by ALZO,
phytosterol esters,
fatty acid triglycerides and their derivatives,
pentaerythritol esters,
esters of dimer diol and dimer diacid, where appropriate, esterified on their alcohol or free acid group(s) by acid or alcohol radicals, in particular dimer dilinoleate esters; such esters can be selected in particular among esters having the following INCI nomenclature: bis-behenyl/isostearyl/phytosteryl dimer dilinoleyl dimer dilinoleate (Plandool G), phytosteryl isostearyl dimer dilinoleate (Lusplan PI-DA, Lusplan PHY/IS-DA), phytosteryl/isosteryl/cetyl/stearyl/behenyl dimer dilinoleate (Plandool H or Plandool S), and mixtures thereof,
mango butter, such as the one marketed under the name Lipex 203 by the AARHUSKARLSHAMN company,
hydrogenated soybean oil, hydrogenated coconut oil, hydrogenated rapeseed oil, mixtures of hydrogenated vegetable oils such as the mixture of hydrogenated vegetable oil from soybean, coconut, palm, and rapeseed, for example the mixture marketed under the name Akogel® by the AARHUSKARLSHAMN company (INCI name Hydrogenated Vegetable Oil),
shea butter, in particular the one whose INCI name is Butyrospermum Parkii Butter, such as the one marketed under the name Sheasoft® by the AARHUSKARLSHAMN company,
cocoa butter, in particular the one marketed under the name CT COCOA BUTTER DEODORIZED by the DUTCH COCOA BV company or the one marketed under the name BURRE DE CACAO NCB HD703 758 by the BARRY CALLEBAUT company;
shorea butter, in particular the one marketed under the name DUB SHOREA T by the STEARINERIE DUBOIS company;
   and mixtures thereof.

Lipophilic Gelling Agents

In addition to waxes, the composition according to the invention may comprise at least one lipophilic gelling agent, for example composed of copolymers of styrene and of olefins such as ethylene, propylene, and/or butylene, optionally combined with silicone-based solvents or hydrocarbon-based solvents, as described in particular in application WO 98/38981 and in U.S. Pat. No. 6,309,629, or copolymers of styrene and of butadiene such as those sold under the name OleoFLEX EG 200 by the Applechem company. They include in particular the gelling agents based on block terpolymers available from the PENRECO company under the trade name VERSAGEL®. Another type of lipophilic gelling agent consists of polyamides such as those identified by the INCI name polyamide-3 and in particular the polymers SYLVACLEAR® AF 1900V and PA 1200V available from the ARIZONA CHEMICAL company as well as those identified by the INCI name “Ethylenediamine/Hydrogenated Dimer Dilinoleate Copolymer Bis-Di-C14-18 Alkyl Amide” and available for example under the trade name SYLVACLEAR® A200V or SYLVACLEAR® A2614V from the ARIZONA CHEMICAL company. The lipophilic gelling agent may alternatively be a hydrophobic modified hectorite or bentone. The gelling agent for the oils may also be a polyurethane gelling agent, preferably of natural origin such as a castor oil derivative available for example under the trade name EstoGel® M by the Polymerexpert company.

Pulverulent Phase

The composition according to the invention also comprises at least one pulverulent phase comprising spherical fillers surface-treated with a metallic soap. The pulverulent phase may also comprise a lamellar filler surface-treated with a metallic soap. The pulverulent phase may preferably also comprise nacres and/or pigments, optionally surface-treated with a metallic soap, and preferably at least nacres, optionally surface-treated with a metallic soap.

Surface Treatment

The pulverulent phase used in the compositions according to the invention is at least partially surface-treated with a metallic soap.

In particular, the pulverulent phase comprises at least spherical fillers surface-treated with a metallic soap.

In particular, the pulverulent phase comprises at least spherical fillers surface-treated with a metallic soap and further comprises a lamellar filler surface-treated with a metallic soap.

According to a preferred embodiment, at least 30% by weight, preferably 30 to 50% by weight, of the nacres is surface-treated with a metallic soap. In this preferred embodiment, the non-volatile oil used is a hydrocarbonated oil.

In particular, the metallic soap is a soap of fatty acids having from 12 to 22 carbon atoms, and in particular from 12 to 18 carbon atoms.

The metal of the metallic soap is preferably selected from zinc and magnesium.

Thus, according to a preferred embodiment, the metallic soap is selected from zinc laurate, magnesium stearate, magnesium myristate, zinc stearate, and mixtures thereof, and preferably the metallic soap is magnesium stearate.

Fillers

The fillers may be inorganic or organic.

The pulverulent phase comprises at least one spherical filler surface-treated with a metallic soap.

The spherical fillers are advantageously selected from:
silica powders;
powders of acrylic (co)polymers, and their derivatives, in particular powders of acrylate (co)polymer, and their derivatives, advantageously selected from a powder of poly(methyl methacrylate), a powder of poly(methyl methacrylate)/ethylene glycol dimethacrylate, a powder of poly(allyl methacrylate)/ethylene glycol dimethacrylate, a powder of ethylene glycol dimethacrylate/lauryl methacrylate copolymer, a powder of acrylate/alkyl acrylate copolymer, optionally crosslinked, hollow particles of expanded acrylonitrile (co)polymer, and mixture(s) thereof;
polyurethane powders;
silicone powders advantageously selected from a powder of polymethylsilsesquioxane, of organopolysiloxane elastomer coated with silicone resin, a powder of organosilicon particles;
polyamide powders, such as Nylon®, in particular Nylon 12;
cellulose powders, such as Cellulobeads D5, D10, D50, and D100 marketed by the Daito company,
   and mixture(s) thereof.

The composition may further comprise a lamellar filler surface-treated with a metallic soap. Among the lamellar fillers, we can cite talc, natural or synthetic mica, certain silicas, clays such as magnesium silicate and aluminum silicate, trimethylsiloxysilicate, kaolin, bentone, calcium carbonate and magnesium bicarbonate, hydroxyapatite, boron nitride, fluorphlogopite, perlite powders, N-Lauroyl-L-lysine powder, sericite, calcium sodium borosilicate, calcium aluminum borosilicate, and mixture(s) thereof.

Among the lamellar fillers, preferred are talc, natural or synthetic mica, certain silicas, clays such as magnesium silicate and aluminum silicate, trimethylsiloxysilicate, kaolin, bentone, calcium carbonate and magnesium bicarbonate, hydroxyapatite, fluorphlogopite, perlite powders, N-Lauroyl-L-Lysine powder, sericite, calcium sodium borosilicate, calcium aluminum borosilicate, and mixture(s) thereof.

The composition may further comprise an additional filler. The additional filler can be selected from inorganic or organic fillers of any form, lamellar, spherical (or hemispherical), regardless of the crystallographic form (for example layer, cubic, hexagonal, orthorombic, etc.)

According to a preferred embodiment, the pulverulent phase comprises lamellar and spherical fillers in a lamellar/spherical ratio ranging from 1/10 to 10/1, preferably from 1/5 to 9/1. This ratio is a weight ratio.

The term “pigments” is understood to mean white or colored particles, inorganic or organic, insoluble in an aqueous medium, intended to color and/or opacify the composition.

The pigments may be white or colored, inorganic and/or organic.

The pigment may be an organic pigment. Organic pigment is understood to mean any pigment which satisfies the definition from the Ullmann encyclopedia in the organic pigment chapter. The organic pigment may in particular be selected from the compounds nitroso, nitro, azo, xanthene, quinoline, anthraquinone, phthalocyanine, of the metal complex type, isoindolinone, isoindoline, quinacridone, perinone, perylene, diketopyrrolopyrrole, thioindigo, dioxazine, triphenylmethane, quinophthalone.

The organic pigment(s) may be selected, for example, among carmine, carbon black, aniline black, melanin, azo yellow, quinacridone, phthalocyanine blue, sorghum red, blue pigments codified in the Color Index under the references CI 42090, 69800, 69825, 73000, 74100, 74160, yellow pigments codified in the Color Index under the references CI 11680, 11710, 15985, 19140, 20040, 21100, 21108, 47000, 47005, green pigments codified in the Color Index under the references CI 61565, 61570, 74260, orange pigments coded in the Color Index under the references CI 11725, 15510,45370, 71105, red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, pigments obtained by oxidative polymerization of indole and phenol derivatives as described in patent FR 2,679,771.

These pigments may also be in the form of composite pigments as described in patent EP 1 184 426. These composite pigments may be composed in particular of particles comprising an inorganic core covered at least partially with an organic pigment and at least one binder ensuring the attachment of the organic pigments to the core.

The pigment may also be a lake. Lake is understood to mean the insolubilized dyes adsorbed on insoluble particles, the ensemble thus obtained remaining insoluble during use. As examples of lakes, we can cite the product known under the following name: D&C Red 7 (CI 15850:1).

The pigment may be an inorganic pigment. Inorganic pigment is understood to mean any pigment which meets the definition from the Ullmann encyclopedia in the inorganic pigment chapter. We can cite, among the inorganic pigments useful in the invention, oxides of zirconium or of cerium, as well as the oxides of zinc, of iron (black, yellow or red)m or of chromium, manganese violet, ultramarine blue, chromium hydrate and ferric blue, titanium dioxide, metallic powders such as aluminum powder and copper powder. The following inorganic pigments can also be used: Ti₂O₅, Ti₃O₅, Ti₂O₃, TiO, ZrO₂ in a mixture with TiCO₂, ZrO₂, Nb₂O₅, CeO₂, ZnS.

The size of the pigment useful in the context of the invention is generally between 10 nm and 10 μm, preferably between 20 nm and 5 μm, and more preferably between 30 nm and 1 μm.

The coloring agent may also be a soluble dye, preferably water-soluble.

Among the water-soluble dyes, we can cite carmine or the products known under the following names: D&C Red 21 (CI 45380), D&C Orange 5 (CI 45370), D&C Red 27 (CI 45410), D&C Orange 10 (CI 45425), D&C Red 3 (CI 45430), D&C Red 4 (CI 15510), D&C Red 33 (CI 17200), D&C Yellow 5 (CI 19140), D&C Yellow 6 (CI 15985), D&C Green (CI 61570), D&C Yellow 1 O (CI 77002), D&C Green 3 (CI 42053), D&C Blue 1 (CI 42090).

The nacres can be selected from those conventionally present in cosmetic products, such as mica/titanium dioxide. Alternatively, this can be nacres based on mica/silica/titanium dioxide, based on synthetic fluorphlogopite/titanium dioxide (SUNSHINE® from MAPRECOS), on calcium sodium borosilicate/titanium dioxide (REFLECKS® from ENGELHARD), or on calcium aluminum borosilicate/silica/titanium dioxide (RONASTAR® from MERCK).

The composition according to the invention comprises from 40 to 85% by weight of pulverulent phase, preferably from 50 to 80% by weight, relative to the total weight of the composition according to the invention.

Polyols

The composition according to the invention may also comprise at least one polyol.

The term “polyol” is understood to mean any organic molecule having in its structure at least 2 free hydroxy groups (—OH). These polyols are preferably liquid at room temperature (25° C.).

As an example, the polyols suitable for use in the composition may be selected from propylene glycol, butylene glycol, pentylene glycol, pentanediol, isoprene glycol, neopentyl glycol, glycerol, polyethylene glycols (PEG) having in particular from 4 to 8 ethylene glycol units, and/or sorbitol.

Preferably, the polyol is glycerol.

According to one particular embodiment, the composition according to the invention comprises from 2 to 30% by weight of polyols, preferably from 5 to 25% by weight, relative to the total weight of the composition.

Emulsifier

The composition according to the invention may also comprise an emulsifier.

These emulsifiers may be selected from nonionic, anionic, cationic, amphoteric surfactants, or polymeric surfactants.

According to one embodiment, the surfactants which can be used in the context of the invention are selected from nonionic surfactants having an HLB value between 8 and 20 at 25° C. We can cite in particular:
esters and ethers of monosaccharides such as the mixture of cetearyl glucoside and cetearyl alcohols such as Montanov 68 from Seppic;
oxyethylene and/or oxypropylene ethers (which may contain from 1 to 150 oxyethylene and/or oxypropylene groups) of glycerol;
oxyethylene and/or oxypropylene ethers (which may contain from 1 to 150 oxyethylene and/or oxypropylene groups) of fatty alcohols (in particular C8-C24 alcohol, and preferably C12-C18) such as polyoxyethylene ether of cetearyl alcohol with 30 oxyethylene groups (CTFA name “Ceteareth-30”), polyoxyethylene ether of stearyl alcohol with 20 oxyethylene groups (CTFA name “Steareth-20”), polyoxyethylene ether of the mixture of C12-C15 fatty alcohols comprising 7 oxyethylene groups (CTFA name “C12-15 Pareth-7”) in particular marketed under the name NEODOL 25-7® by SHELL CHEMICALS;
fatty acid esters (in particular of C8-C24 acid, and preferably C16-C22) and of polyethylene glycol (which may comprise from 1 to 150 ethylene glycol units) such as PEG-50 stearate and PEG-40 monostearate in particular, marketed under the name MYRJ 52P® by the ICI UNIQUEMA company, or PEG-30 glyceryl stearate in particular marketed under the name TAGAT S® by the Evonik GOLDSCHMIDT company;
fatty acid esters (in particular of C8-C24 acid, and preferably C16-C22) and oxyethylene and/or oxypropylene glycerol ethers (which may contain from 1 to 150 oxyethylene and/or oxypropylene groups), such as PEG-200 glyceryl stearate, in particular sold under the name Simulsol 220 TM® by the SEPPIC company; PEGylated glyceryl stearate with 30 ethylene oxide groups such as the TAGAT S® product sold by the Evonik GOLDSCHMIDT company, PEGylated glyceryl oleate with 30 ethylene oxide groups such as the TAGAT O® product sold by the Evonik GOLDSCHMIDT company, PEGylated glyceryl cocoate containing 30 ethylene oxide groups such as the VARIONIC LI 13® product sold by the SHEREX company, PEGylated glyceryl isostearate containing 30 ethylene oxide such as the TAGAT L® product sold by the Evonik GOLDSCHMIDT company, and PEGylated glyceryl laurate with 30 ethylene oxide groups such as the product TAGAT I® from the company Evonik GOLDSCHMIDT,
fatty acid esters (in particular of C8-C24 acid, and preferably C16-C22 acid) and oxyethylene and/or oxypropylene sorbitol ethers (which may contain from 1 to 150 oxyethylene and/or oxypropylene groups), such as polysorbate 20 in particular sold under the name Tween 20® by the CRODA company, polysorbate 60 in particular sold under the name Tween 60® by the CRODA company,
dimethicone copolyol, such as the one sold under the name 02-5220® by the DOW CORNING company,
dimethicone copolyol benzoate (FINSOLV SLB 101® and 201® from the FINTEX company),
copolymers of propylene oxide and ethylene oxide, also called EO/PO polycondensates,
lysophospholipids, in particular lysophosphatidylcholine of the following formula [CHEM1]:

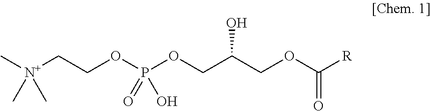

where R is a fatty acid chain, comprising in particular from 10 to 25 carbon atoms, preferably from 15 to 20. Preferably, the lysophospholipid used in the composition of the invention is obtained from soybeans. More preferably, its INCI name is Glycine soja (soybean) seed extract. For example, use is made of a mixture of 80% by weight of glycerin and 20% by weight of Glycine soja (soybean) seed extract marketed by Kemin under the name Lysofix Liquid®;
emulsifying waxes such as the self-emulsifying wax sold under the name Polawax NF by Croda, or the PEG-8 beeswax sold under the name Apifil by Gattefossé, and mixtures thereof.

According to a preferred embodiment, the emulsifier having an HLB value between 8 and 20 is selected from fatty acid esters and oxyethylene and/or oxypropylene sorbitol ethers, and mixtures thereof.

Lysophospholipids such as Lysofix Liquid® allow thickening the composition.

According to one embodiment, the surfactants which can be used in the composition according to the invention are selected from nonionic surfactants having an HLB of less than or equal to 8 at 25° C. We can cite in particular:
esters and ethers of monosaccharides such as sucrose stearate, sucrose cocoate, sorbitan stearate, and mixtures thereof, such as Arlatone 2121® marketed by the ICI company;
oxyethylene and/or oxypropylene ethers (which may contain from 1 to 150 oxyethylene and/or oxypropylene groups) of fatty alcohols (in particular of C8-C24 alcohol, and preferably C12-C18) such as the oxyethylene ether of stearyl alcohol with 2 oxyethylene groups (CTFA name “Steareth-2”);
esters of fatty acids (in particular of C8-C24 acid, and preferably C16-C22) and of polyol, in particular of glycerol or sorbitol, such as glyceryl stearate, such as the product sold under the name TEGIN M® by the Evonik GOLDSCHMIDT company, glyceryl laurate such as the product sold under the name IMWITOR 312® by the HULS company, polyglyceryl-2 stearate, polyglyceryl-2 triisostearate, sorbitan tristearate, glyceryl ricinoleate;
lecithins, such as soybean lecithins (such as Emulmetik 100 J from Cargill, or Biophilic H from Lucas Meyer);
silicone emulsifiers.

The silicone emulsifier that can be used in the composition according to the invention is a siloxane polymer comprising:
a fatty side chain,
an oxyethylene or oxypropylene side chain and/or a polyethoxylated side chain (=glyceryl), and optionally
a silicone side chain.

The fatty side chain of the silicone emulsifier allows good compatibility with the fatty phase of the water-in-oil emulsion. The silicone side chain makes it possible to have good compatibility with non-volatile siliconated oil, when the non-volatile oil of the cosmetic emulsion of the invention is a siliconated oil. According to one embodiment of the invention, the silicone emulsifier comprises a fatty side chain and a silicone side chain.

More particularly according to the invention, the silicone emulsifier is selected from the group comprising:
the compound of the following formula [CHEM2] in which w is an integer ranging from 1 to 1000, x′ is an integer ranging from 1 to 50, x, y, and z represent, independently of each other, an integer ranging from 1 to 100:

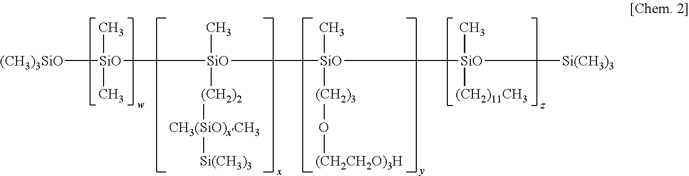

the compound of the following formula [CHEM3] in which x1 is an integer ranging from 1 to 1000, w1 is an integer ranging from 1 to 50, y1 and z1 represent, independently of each other, an integer ranging from 1 to 100:

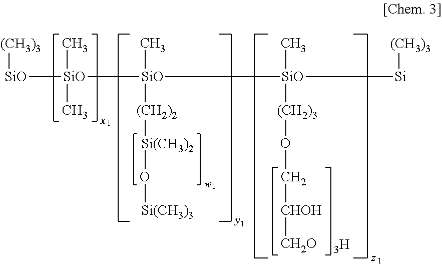

the compound of the following formula [CHEM4] in which w2 is an integer ranging from 1 to 1000, v2 is an integer ranging from 1 to 50, x2, y2, and z2 represent, independently of each other, an integer ranging from 1 to 100:

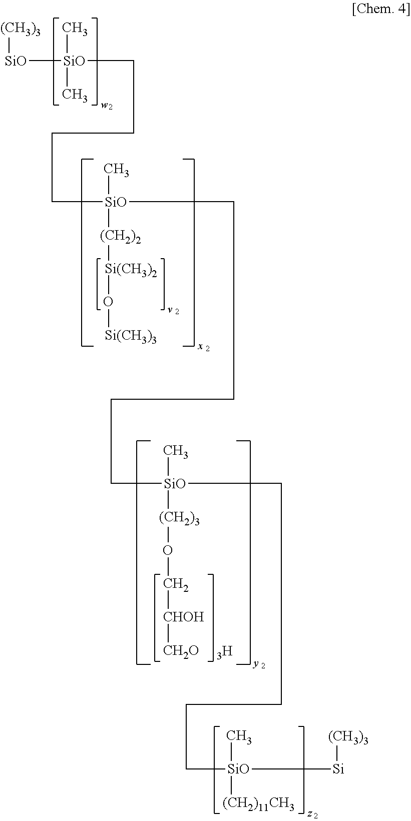

the mixture of cyclomethicone/dimethicone copolyol sold under the name 02-3225C® by the DOW CORNING company,

and mixtures thereof.

According to one particular embodiment, the silicone emulsifier is selected from the group comprising the siloxane polymers marketed by the SHIN-ETSU company under the references KF6038, KF6104, KF6105, KF6106, and mixtures thereof.

The compound KF6038, having the INCI name “Lauryl PEG-9 Polydimethylsiloxyethyl Dimethicone”, corresponds to general formula (I). This siloxane polymer comprises a silicone side chain, an oxyethylene side chain, and a fatty side chain (lauryl).

The compound KF6104, having the INCI name “Polyglyceryl-3 Polydimethylsiloxyethyl Dimethicone”, and the compound KF6106, having the INCI name “Polyglyceryl-3 Polydimethylsiloxyethyl Dimethicone”, correspond to general formula (II). This siloxane polymer comprises a silicone side chain and a glyceryl side chain.

The compound KF6105, having the INCI name “Lauryl Polyglyceryl-3 Polydimethylsiloxyethyl Dimethicone”, corresponds to general formula (III). This siloxane polymer comprises a silicone side chain, a glyceryl side chain, and a fatty side chain (lauryl).

In a preferred embodiment, in particular when the composition comprises siliconated oils, the surfactant is selected from silicone surfactants such as compound KF6028 or compound KF6038 or a mixture thereof.

The silicone emulsifier is present in the cosmetic composition of the invention in a content ranging from 0.1% to 5%, preferably from 1% to 3%, the percentages being percentages by weight relative to the total weight of the composition.

In a preferred embodiment, in particular when the composition comprises hydrocarbonated oils, the surfactant is selected from non-silicone surfactants, preferably polysorbate 20.

The composition according to the invention may contain from 0.1 to 5% by weight of emulsifier, relative to the total weight of said composition, preferably from 1 to 3% by weight.

Film-Forming Polymer

The composition according to the invention may also comprise at least one film-forming polymer.

Among the film-forming polymers which can be used in the compositions of the invention, we can cite synthetic polymers, of the free-radical or polycondensate type, polymers of natural origin, and mixtures thereof.

The term “free-radical film-forming polymer” means a polymer obtained by polymerization of unsaturated monomers, in particular ethylenically, each monomer being capable of homopolymerization (unlike polycondensates).

The free-radical type of film-forming polymers can in particular be vinyl polymers or copolymers, in particular acrylic polymers.

The vinyl film-forming polymers can result from polymerization of ethylenically unsaturated monomers having at least one acid group and/or esters of these acidic monomers and/or amides of these acidic monomers.

For the monomer carrying an acid group, it is possible to use α, β-ethylenic unsaturated carboxylic acids such as acrylic acid, methacrylic acid, crotonic acid, maleic acid, itaconic acid. Use is preferably made of (meth)acrylic acid, itaconic acid, and crotonic acid, and more preferably itaconic acid (for example a metal salt of poly(itaconic acid) such as the one marketed under the trade reference REVCARE NE 100S by the Itaconix company).

The esters of acidic monomers are advantageously selected from esters of (meth)acrylic acid (also called (meth)acrylates), in particular alkyl (meth)acrylates, in particular C1-C30 alkyl, preferably C1-C20, aryl (meth)acrylates, in particular C6-C10 aryl, hydroxyalkyl (meth)acrylates, in particular C2-C6 hydroxyalkyl.

Among the alkyl (meth)acrylates, we can cite methyl methacrylate, ethyl methacrylate, butyl methacrylate, isobutyl methacrylate, 2-ethylhexyl methacrylate, lauryl methacrylate, cyclohexyl methacrylate.

Among the hydroxyalkyl (meth)acrylates, we can cite hydroxyethyl acrylate, 2-hydroxypropyl acrylate, hydroxyethyl methacrylate, and 2-hydroxypropyl methacrylate.

Among the aryl (meth)acrylates, we can cite benzyl acrylate and phenyl acrylate.

Particularly preferred esters of (meth)acrylic acid are the alkyl (meth)acrylates.

According to the invention, the alkyl group of the esters can be either fluorinated or perfluorinated, meaning that some or all of the hydrogen atoms of the alkyl group are substituted by fluorine atoms.

For the amides of acidic monomers, we can cite, for example, (meth)acrylamides, and in particular N-alkyl (meth)acrylamides, in particular C2-C12 alkyl. Among the N-alkyl (meth)acrylamides, we can cite N-ethylacrylamide, N-tert-butylacrylamide, N-tert-octylacrylamide, and N-undecylacrylamide.

The vinyl film-forming polymers may also result from the homopolymerization or copolymerization of monomers selected from vinyl esters and styrene monomers. In particular, these monomers can be polymerized with acidic monomers and/or their esters and/or their amides, such as those mentioned above.

As examples of vinyl esters, we can cite vinyl acetate, vinyl neodecanoate, vinyl pivalate, vinyl benzoate, and vinyl tert-butylbenzoate.

We can also cite, for the styrene monomers, styrene and alpha-methylstyrene.

We can also cite the styrene-butadiene block copolymers such as the products from the Kraton company, or OLEOFLEX EG 200 from the APPLECHEM company.

Among the film-forming polycondensates, we can cite polyurethanes, polyesters, polyester amides, polyamides, and epoxy ester resins, polyureas.

The polyurethanes can be selected from anionic, cationic, nonionic or amphoteric polyurethanes, acrylic polyurethanes, polyurethane-polyvinylpirrolidones, polyester-polyurethanes, polyether-polyurethanes, polyureas, polyurea-polyurethanes, and mixtures thereof.

The polyesters can be obtained, in a known manner, by polycondensation of dicarboxylic acids with polyols, in particular diols.

The dicarboxylic acid can be aliphatic, alicyclic, or aromatic. As examples of such acids, we can mention: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornene dicarboxylic acid, diglycolic acid, thiodipropionic acid, 2,5-naphthalenedicarboxylic acid, 2,6-naphthalenedicarboxylic acid. These dicarboxylic acid monomers can be used alone or in a combination of at least two dicarboxylic acid monomers. Among these monomers, phthalic acid, isophthalic acid, and terephthalic acid are preferably selected.

The diol can be selected from the aliphatic, alicyclic, and aromatic diols. A diol selected from: ethylene glycol, diethylene glycol, triethylene glycol, 1,3-propanediol, cyclohexanedimethanol, and 4-butanediol is preferably used. For other polyols, glycerol, pentaerythritol, sorbitol, trimethylolpropane can be used.

Polyester amides can be obtained in a manner analogous to polyesters, by polycondensation of diacids with diamines or amine alcohols. For the diamine, ethylenediamine, hexamethylenediamine, meta- or para-phenylenediamine can be used. For the amine alcohol, monoethanolamine can be used. For polyamide resins, we can also cite the one corresponding to the INCI name DIISOSTEARYL MALATE & BIS-DIOCTADECYLAMIDE DIMER DILINOLEIC ACID/ETHYLENEDIAMINE COPOLYMER marketed under the name Haimalate PAM by the Kokyu Alcohol Kogyo company.

The polyester may further comprise at least one monomer bearing at least one —SO3M group, with M representing a hydrogen atom, an ammonium ion NH4+ or a metal ion, for example such as an ion Na+, Li+, K+, Mg2+, Ca2+, Cu2+, Fe2+, Fe3+. It is possible in particular to use a bifunctional aromatic monomer comprising such a —SO3M group.

The aromatic nucleus of the bifunctional aromatic monomer further bearing an —SO3M group as described above can be selected, for example, from the benzene, naphthalene, anthracene, diphenyl, oxidiphenyl, sulfonyldiphenyl, methylene diphenyl rings. As an example of a bifunctional aromatic monomer also bearing an —SO3M group, we can mention: sulfoisophthalic acid, sulfoterephthalic acid, sulfophthalic acid, 4-sulfonaphthalene-2,7-dicarboxylic acid.

It is possible to use copolymers based on isophthalate/sulfoisophthalate, and more particularly copolymers obtained by condensation of diethylene glycol, cyclohexanedimethanol, isophthalic acid, sulfoisophthalic acid.

The polymers of natural origin, optionally modified, can be selected from shellac resin, sandarac gum, gum arabic (ACACIA SENEGAL GUM), dammars, elemis, copals, cellulose polymers, polymer extracts from the fruit of Caesalpinia spinosa and/or from the algae Kappaphycus alvarezii (such as the Filmexel® product marketed by the Silab company), and mixtures thereof. A natural polymer such as Filmexel® makes it possible in particular to improve the longevity of the film obtained from the composition according to the invention. We can also cite the film-forming polymers corresponding to the INCI name SHOERA ROBUSTA RESIN+BEESWAX, SHOERA ROBUSTA RESIN+SUNFLOWER OIL, ARAUCARIA+SUNFLOWER OIL, ARAUCARIA+CASTOR OIL, SHOERA ROBUSTA+OCTYLDODECANOL

According to one embodiment, the film-forming polymer can be a polymer dissolved in a liquid fatty phase comprising organic solvents or oils (the film-forming polymer is then said to be a fat-soluble polymer).

As an example of a fat-soluble polymer, we can cite vinyl ester copolymers (the vinyl group being directly linked to the oxygen atom of the ester group and the vinyl ester having a saturated hydrocarbon radical, linear or branched, of 1 to 19 carbon atoms, bonded to the carbonyl of the ester group) and at least one other monomer which may be a vinyl ester (different from the vinyl ester already present), an α-olefin (having from 8 to 28 carbon atoms), an alkyl vinyl ether (whose alkyl group contains from 2 to 18 carbon atoms), or an allylic or methallyl ester (having a saturated, linear or branched hydrocarbon radical, of 1 to 19 carbon atoms, linked to the carbonyl of the ester group).

These copolymers can be crosslinked using crosslinkers which can be either of the vinyl type or of the allyl or methallyl type, such as tetra(allyloxy)ethane, divinylbenzene, divinyl octanedioate, divinyl dodecanedioate, and divinyl octadecanedioate.

As examples of these copolymers, we can cite the copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyl octanoate/vinyl laurate, allyl 2,2-dimethyl pentanoate/vinyl laurate, vinyl dimethyl propionate/vinyl stearate, allyl dimethyl propionate/vinyl stearate, vinyl propionate/vinyl stearate, crosslinked with 0.2% divinylbenzene, vinyl dimethyl propionate/vinyl laurate, crosslinked with 0.2% divinylbenzene, vinyl acetate/octadecyl vinyl ether, crosslinked with 0.2% tetra(allyloxy)ethane, vinyl acetate/allyl stearate crosslinked with 0.2% divinylbenzene, vinyl acetate/1-octadecene crosslinked with 0.2% divinylbenzene, and allyl propionate/allyl stearate crosslinked with 0.2% divinylbenzene.

For the fat-soluble film-forming polymers, we can also cite fat-soluble copolymers, and in particular those resulting from copolymerization of vinyl esters having from 9 to 22 carbon atoms or of alkyl acrylates or methacrylates, the allyl radicals having from 10 to 20 carbon atoms.

Such fat-soluble copolymers can be selected from copolymers of vinyl polystearate, of vinyl polystearate crosslinked with the aid of divinylbenzene, diallyl ether or diallyl phthalate, copolymers of stearyl poly(meth)acrylate, of polyvinyl laurate, of lauryl poly(meth)acrylate, these poly(meth)acrylates able to be crosslinked using methylene glycol dimethacrylate or tetraethylene glycol dimethacrylate.

The fat-soluble copolymers defined above are known and in particular are described in application FR-A-2232303; they can have an average molecular weight ranging from 2,000 to 500,000 and preferably from 4,000 to 200,000.

We can also cite fat-soluble homopolymers, and in particular those resulting from homopolymerization of vinyl esters having from 9 to 22 carbon atoms or of alkyl acrylates or methacrylates, the alkyl radicals having from 2 to 24 carbon atoms.

As examples of fat-soluble homopolymers, we can cite in particular: polyvinyl laurate and lauryl poly(meth)acrylates, these poly(meth)acrylates possibly being crosslinked using ethylene glycol dimethacrylate or tetraethylene glycol dimethacrylate.

For the fat-soluble film-forming polymers which can be used in the invention, we can also cite polyalkylenes and in particular copolymers of C2-C20 alkenes, such as polybutene, alkyl celluloses with a linear or branched alkyl radical, saturated or unsaturated at C1 to C8 such as ethyl cellulose and propyl cellulose, copolymers of vinylpyrrolidone (VP) and in particular copolymers of vinylpyrrolidone and C2 to C40 alkene and better still C3 to C20 alkene. As an example of a copolymer of VP which can be used in the invention, we can cite the copolymer of VP/vinyl acetate, VP/ethyl methacrylate, butylated polyvinylpyrolidone (PVP), VP/ethyl methacrylate/methacrylic acid, VP/eicosene (ANTARON V220 marketed by the Ashland company), VP/hexadecene (ANTARON V216 marketed by the Ashland company), VP/triacontene, VP/styrene, VP/acrylic acid/lauryl methacrylate.

We can also cite dextrin esters and in particular:
dextrin isostearate & isostearic acid marketed under the name UNIFILMA HVY by the Chiba Flour Milling company,
dextrin palmitate/ethyl hexanoate marketed under the name RHEOPEARL TT by the Chiba Flour Milling company,
Dextrin Myristate marketed under the name RHEOPEARL MKL2 by the Chiba Flour Milling company.

We can also cite sugar esters and in particular sucrose acetate isobutyrate marketed under the name EASTMAN SUSTANE SAIB by the EASTMAN company.

We can also cite silicone resins, generally soluble or swellable in silicone oils, which are crosslinked polyorganosiloxane polymers. The nomenclature of silicone resins is known under the name “MDTQ”, the resin being described according to the various siloxane monomer units that it comprises, each of the letters “MDTQ” characterizing a type of unit.

As examples of commercially available polymethylsilsesquioxane resins, we can cite those marketed by the Wacker company under the reference Resin MK such as Belsil PMS MK, and by the SHIN-ETSU company under the references KR-220L, or Silform flexible resin.

For the siloxysilicate resins, we can cite trimethylsiloxysilicate (TMS) resins such as those marketed under the reference SR1000 by the General Electric company or under the reference TMS 803 by the Wacker company. We can also cite the trimethylsiloxysilicate resins marketed in a solvent such as cyclomethicone, sold under the name “KF-7312J” by the Shin-Etsu company, “DOWSIL™ RSN-0749”, “DOWSIL™ 593 Fluid” by the Dow Corning company.

We can also cite copolymers of silicone resins such as those cited above with polydimethylsiloxanes, such as the pressure-sensitive adhesive copolymers marketed by the Dow Corning company under the reference BIO-PSA and described in document U.S. Pat. No. 5,162,410, or silicone copolymers resulting from the reaction of a silicone resin, such as those described above, and of a diorganosiloxane as described in document WO 2004/073626.

It is also possible to use copolymers with a non-silicone organic backbone grafted with monomers containing a polysiloxane unit, for example such as the butyl acrylate/hydroxypropyl dimethicone acrylate copolymer marketed under the name GRANACRYSIL BAS by the GRANT company.

Lastly, we can cite the acrylates/polytrimethylsiloxymethacrylate copolymers comprising a carbosiloxane dendrimer structure grafted onto a vinyl backbone, available commercially under the references DOW CORNING FA 4002 ID or DOW CORNING FA 4001 CM.

It is also possible to use silicone polyamides of the polyorganosiloxane type such as those described in documents U.S. Pat. Nos. 5,874,069, 5,919,441, 6,051,216, and 5,981,680.

Silicone Elastomers

The composition according to the invention may also comprise a silicone elastomer.

Among these, we can cite the at least partially crosslinked polymers resulting from the reaction of an organopolysiloxane bearing unsaturated groups, such as vinyl or allyl groups, located at the end or in the middle of the chain, preferably on a silicon atom, with another reactive silicone compound such as an organohydrogenpolysiloxane. These polymers are usually available in the form of a gel in a volatile or non-volatile silicone solvent or in a hydrocarbon solvent. Examples of such elastomers are marketed in particular by the SHIN ETSU company under the trade names KSG-6, KSG-16, KSG-31, KSG-32, KSG-41, KSG-42, KSG-43, and KSG-44, and by the DOW CORNING company under the trade names DOWSIL™ 9040 and DOWSIL™ 9041. Another oily gelling agent consists of a silicone polymer, obtained by self-polymerization of an organopolysiloxane functionalized with epoxy and hydrosilyl groups, in the presence of a catalyst, which is commercially available from the GENERAL ELECTRIC company under the trade name VELVESIL® 125. Another lipophilic gelling agent consists of a cyclic dimethicone/vinyldimethicone copolymer such as the one marketed by the JEEN company under the trade name JEESILC® PS (including PS-VH, PS-VHLV, PS-CM, PS-CMLV and PS-DM).

According to a preferred embodiment, the silicone elastomer can be an emulsifier, preferably selected from polyoxyalkylenated and polyglycerolated silicone elastomers.

For the polyoxyalkylenated silicone elastomers, we can cite those described in U.S. Pat. Nos. 5,236,986, 5,412,004, 5,837,793, 5,811,487.

For the polyoxyalkylenated silicone elastomers, the following can be used: those with the INCI name PEG-10 Dimethicone/Vinyl Dimethicone Crosspolymer: such as those marketed under the names “KSG-21”, “KSG-20”, by Shin Etsu; those with the INCI name PEG-15 Lauryl Dimethicone/Vinyl Dimethicone Crosspolymer: such as those marketed under the names “KSG-30” and “KSG-31”, “KSG-32” (in isododecane), “KSG-33” (in trioctanoin), “KSG-210”, “KSG-310” (in mineral oil), “KSG-320” (in isododecane), “KSG-330”, “KSG-340”, by the Shin Etsu company.

For the polyglycerolated silicone elastomers, the following can be used: those with the INCI name Dimethicone (and) Dimethicone/Polyglycerin-3 Crosspolymer: such as those sold under the names “KSG-710” by Shin Etsu; those with the INCI name Lauryl Dimethicone/Polyglycerin-3 Crosspolymer: such as those sold under the names “KSG-840” (in squalene) by the Shin Etsu company.

Aqueous Phase

The composition according to the invention may also comprise an aqueous phase comprising water and optionally at least one solvent soluble in water other than the polyols described above.

In the invention, the term “solvent soluble in water” denotes a compound which is liquid at room temperature and water-miscible (miscibility in water greater than 50% by weight at 25° C. and atmospheric pressure).

The solvents soluble in water which can be used in the compositions according to the invention can be volatile.

Among the solvents soluble in water which can be used in the compositions in accordance with the invention, we can cite in particular the mono-alcohols having from 1 to 5 carbon atoms such as ethanol and isopropanol, and C₃-C₄ ketones and C₂-C₄ aldehydes.

According to a preferred embodiment, the composition according to the invention is free of water.

Cosmetic Active Ingredient

The composition according to the invention may also comprise at least one cosmetic active ingredient, which can be selected from the group consisting of vitamins, antioxidants, moisturizing agents, anti-pollution agents, keratolytic agents, astringents, anti-inflammatory agents, whitening agents, sunless tanners, and agents promoting microcirculation.

Examples of vitamins include vitamins A, B1, B2, B6, C, and E, and their derivatives, pantothenic acid and its derivatives, and biotin.

Examples of antioxidants include ascorbic acid and its derivatives such as ascorbyl palmitate, ascorbyl tetraisopalmitate, ascorbyl glucoside, magnesium ascorbyl phosphate, sodium ascorbyl phosphate, and ascorbyl sorbate; tocopherol and its derivatives, such as tocopherol acetate, tocopherol sorbate, and other tocopherol esters; BHT and BHA; esters of gallic acid, phosphoric acid, citric acid, maleic acid, malonic acid, succinic acid, fumaric acid, cephalin, hexametaphosphate, phytic acid, and plant extracts, for example roots of Zingiber Officinale (ginger) such as Blue Malagasy Ginger marketed by the BIOLANDES company, Chondrus crispus, Rhodiola, Thermus thermophilus, Yerba mate leaf, oak wood, Kayu Rapet bark, Sakura leaves, and ylang ylang leaves.

Examples of moisturizers include polyethylene glycol, propylene glycol, dipropylene glycol, glycerin, butylene glycol, xylitol, sorbitol, maltitol, mucopolysaccharides, such as chondroitin sulfuric acid, high or low molecular weight hyaluronic acid or alternatively hyaluronic acid potentiated by a silanol derivative such as the active ingredient Epidermosil® marketed by the Exymol company, and mucoitinsulphuric acid; caronic acid; atelo collagen; chloresteryl-12-hydroxystearate; bile salts, a major component of NMF (natural moisturizing factor) such as a salt of pyrrolidone carboxylic acid and a salt of lactic acid, an amino acid analog such as urea, cysteine, and serine; short chain soluble collagen, PPG diglycerins, 2-methacryloyloxyethyl phosphorylcholine homo- and copolymers such as Lipidure HM and Lipidure PMB from NOF; allantoin; glycerin derivatives such as PEG/PPG/Polybutylene Glycol-8/5/3 Glycerin from NOF sold under the trade name Wilbride®S753, or alternatively glyceryl-polymethacrylate from Sederma sold under the trade name Lubrajel®MS; trimethylglycine sold under the trade name Aminocoat® by the Ashahi Kasei Chemicals company, and various plant extracts such as Castanea sativa extracts, hydrolyzed hazelnut proteins, Polyanthes Tuberosa polysaccharides, Argania spinosa kernel oil, and nacre extracts containing a conchiolin which are sold in particular by the Maruzen company (Japan) under the trade name Pearl Extract®.

Other examples of moisturizers include compounds stimulating the expression of maritriptase MT-SP1, such as carob pulp extract, as well as agents stimulating the expression of CERT, rNAT2, or of FN3K or FN3K-RP; agents increasing the proliferation or differentiation of keratinocytes, either directly or indirectly by stimulating, for example, the production of β-endorphins, such as extracts of Thermus thermophilus or extracts of Theobroma cacao hulls, water-soluble extracts of corn, peptide extracts of Voandzeia subterranea and niacinamide; epidermal lipids and agents increasing the synthesis of epidermal lipids, either directly or by stimulating certain β-glucosidases which modulate the deglycosylation of lipid precursors such as glucosylceramide into ceramides, such as phospholipids, ceramides, lupine protein hydrolysates, and derivatives of dihydrojasmonic acid.

Examples of anti-pollution agents include Moringa pterygosperma seed extract (for example Purisoft® from LSN); shea butter extract (for example Detoxyl® from Silab), a mixture of ivy extract, of phytic acid, of sunflower seed extract (for example Osmopur® from Sederma).

Examples of keratolytic agents include α-hydroxy acids (for example glycolic, lactic, citric, malic, mandelic, or tartaric acids) and β-hydroxy acids (for example salicylic acid), and their esters, such as C12-13 alkyl lactates, and plant extracts containing these hydroxy acids, such as Hibiscus sabdariffa extracts.

Examples of astringents include witch hazel extracts.

Examples of anti-inflammatory agents include bisabolol, allantoin, tranexamic acid, zinc oxide, sulfur oxide and its derivatives, chondroitin sulfate, glycyrrhizic acid and its derivatives such as glycyrrhizinates.

Examples of whitening agents include arbutin and its derivatives, ferulic acid (such as Cytovector®: water, glycol, lecithin, ferulic acid, hydroxyethyl cellulose, marketed by BASF) and its derivatives, kojic acid, resorcinol, lipoic acid and its derivatives such as resveratrol diacetate monolipoate as described in patent application WO2006134282, ellagic acid, leucodopachrome and its derivatives, vitamin B3, linoleic acid and its derivatives, ceramides and their homologues, a peptide as described in patent application WO2009010356, a bioprecursor as described in patent application WO2006134282 or a tranexamate salt such as the hydrochloride salt of cetyl tranexamate, a licorice extract (Glycyrrhiza glabra extract), which is sold in particular by the Maruzen company under the trade name Licorice Extract®, a whitening agent also having an antioxidant effect, such as vitamin C compounds, including mineral ascorbates, ascorbyl esters of fatty acids or of sorbic acid, and other ascorbic acid derivatives, for example ascorbyl phosphates, such as magnesium ascorbyl phosphate and sodium ascorbyl phosphate, or esters of ascorbic acid saccharides, which include, for example, ascorbyl-2-glucoside, 2-O-alpha-D-glucopyranosyl-L-ascorbate, or 6-O-beta-D-galactopyranosyl-L-ascorbate. An active ingredient of this type is sold in particular by the DKSH company under the trade name Ascorbyl Glucoside®.

An example of a sunless tanner is DHA.

Examples of agents promoting microcirculation include an extract of lupine (such as Eclaline® from Silab), of ruscus, of horse chestnut, of ivy, of ginseng, or of sweet clover, caffeine, nicotinate and its derivatives, an algae extract of Corallina officinalis such as the one marketed by CODIF; and mixtures thereof. These agents, which act on the skin's microcirculation, can be used to prevent dulling of the complexion and/or to improve uniformity and radiance of the complexion.

Additives

The composition according to the invention may comprise other ingredients as long as they do not interfere with the desired properties of the composition. These other ingredients may, for example, be preservatives, pH adjusters such as citric acid or arginine, antimicrobial agents, perfumes, sunscreens, and mixtures thereof.

Preparation Process

An object of the invention is also a process for preparing a solid cosmetic composition according to the invention, comprising:
premixing the powders constituting the pulverulent phase
preparating a fatty binder comprising the non-volatile liquid fatty phase
mashing the powders in with the fatty binder by extrusion, and
shaping the composition by pressing.

Method for Making Up Keratinous Elements

The invention also relates to a method for making up the skin or lips, consisting of applying a solid cosmetic composition according to the invention to the skin or lips.

### EXAMPLES

### Example 1: Eye Shadows

Solid eye shadows were prepared, having the composition shown in the following Table 1.

TABLE 1

| | Content (% by weight) | | | | | |
|---|---|---|---|---|---|---|
| | EYESH. | EYESH. | EYESH. | EYESH. | EYESH. | EYESH. |
| INCI standard | 1 | 2 | 3 | 4 | 5 | 6 |
| Coated inorganic | 7.46 | | | | | |
| pigments | | | | | | |
| Organic pigments | 2.39 | | | | | |
| Nacres (uncoated) | | | | 40 | 30.80 | 40 |
| Nacres coated with | 34.76 | 39.00 | 39.00 | | | |
| magnesium stearate | | | | | | |
| Lamellar fillers coated | 19.20 | | | | | |
| with other silicone | | | | | | |
| coating (hydrogen | | | | | | |
| dimethicone (SiO1-5) | | | | | | |
| Lamellar fillers coated | | 19.50 | 19.50 | 30.00 | 27.24 | 12.30 |
| with magnesium | | | | | | |
| stearate | | | | | | |
| Spherical fillers | 7.97 | 6.5 | 6.5 | 10.00 | 7.70 | 16.00 |
| (Cellulobeads) coated | | | | | | |
| with magnesium | | | | | | |
| stearate | | | | | | |
| Styrene butadiene | 1.39 | 0.26 | | | | |
| copolymer | | | | | | |
| (1.75% OLEOFLEX | | | | | | |
| EG200) | | | | | | |
| Dextrin isostearate & | | | 3.50 | | | |
| isostearic acid | | | | | | |
| (Unifilma HVY) | | | | | | |
| Butyl acrylate/ | | | 2.10 | 6.00 | 4.62 | 4.79 |
| hydroxypropyl | | | | | | |
| dimethicone acrylate | | | | | | |
| copolymer (Granacrysil | | | | | | |
| BAS) | | | | | | |
| Castor oil/IPDI | 2.80 | 2.80 | 2.80 | | | |
| copolymer (3.5% | | | | | | |
| EstoGel M) | | | | | | |
| Dimethicone | | | | | 0.23% | 0.24% |
| crosspolymer from | | | | | (1.54%) | (1.60%) |
| DOWSIL 9041 | | | | | | |
| (% DOWSIL 9041) | | | | | | |
| Microcristalline wax | 1.23 | 1.54 (7.7) | 1.33 | | | |
| (from petroleum jelly) | (6.14)) | | (6.65) | | | |
| (% petroleum jelly) | | | | | | |
| Dimethicone (from | | | | | 1.30 | 1.36 |
| DOWSIL 9041) | | | | | | |
| Dimethicone (KF96A- | | | | | 23.00 | 20.00 |
| 10CS) | | | | | | |
| Dimethicone (Xiameter | 5.03 | 6.30 | 6.30 | 4.00 | | |
| PMX-200 fluid 350 cS) | | | | | | |
| Ethylhexyl | 5.58 | 7.00 | 7.00 | 3.00 | 2.31 | 2.40 |
| polyhydroxystearate | | | | | | |
| (Dub Estoline) | | | | | | |
| Liquid paraffin | 4.30 | 5.39 | 4.65 | | | |
| Dicaprylyl carbonate | 2.79 | 3.50 | 1.75 | | | |
| Helianthus annuus | 0.63 | 0.78 | | | | |
| seed oil (from | | | | | | |
| OLEFLEX EG 200) | | | | | | |
| Caprylic/capric | 5.30 | 6.65 | 4.90 | | | |
| triglycerides (from | | | | | | |
| Miglyol 812N and | | | | | | |
| Estogel M or | | | | | | |
| OLEOFLEX EG 200) | | | | | | |
| PEG-9 | | | | 2 | 1.54 | 1.60 |
| polydimethylsiloxyethyl | | | | | | |
| dimethicone (KF6028) | | | | | | |

The powders constituting the pulverulent phase were premixed.

The fatty binder comprising the fatty phase (liquid and solid) was then prepared.

The powders were mashed in with the fatty binder by extrusion.

The compositions were then shaped by pressing.

The eye shadows obtained exhibit good cohesion and good impact resistance. In particular, they can be stored and transported without crumbling or cracking. Their texture allows easy pick-up and application.

### Example 2: Blush—Complexion Highlighter—Bronzer—Lipstick

Solid compositions were prepared having the composition presented in the following Table 2.

TABLE 2

| | Content (% by weight) | | | |
|---|---|---|---|---|
| | | Complexion | | Lip- |
| INCI standard | Blush | highlighter | Bronzer | stick |
| Inorganic pigments (uncoated) | 13.65 | | 0.5 | |
| Coated inorganic pigments | | | 6.17 | |
| Organic pigments | | | | 8.55 |
| Nacres (uncoated) | 3.60 | 42.60 | 4.50 | |
| Nacres coated with magnesium | | 6.83 | | |
| stearate | | | | |
| Lamellar fillers coated with | | | 38.17 | 15.07 |
| another hydrogen dimethicone | | | | |
| coating (SiO1-5) | | | | |
| Lamellar fillers coated with | 52.28 | | 11.8 | 44.71 |
| magnesium stearate | | | | |
| Spherical fillers (Cellulobeads) | 6.30 | 13.36 | 8.00 | 8.89 |
| coated with magnesium stearate | | | | |
| Magnesium stearate as | | 2.94 | | |
| compacting agent | | | | |
| Butyl acrylate/hydroxypropyl | | | 1.60 | 4.44 |
| dimethicone acrylate copolymer | | | | |
| (Granacrysil BAS) | | | | |
| Dimethicone crosspolymer | 0.67 | | 0.47 | 0.22 |
| from DOWSIL 9041 | (4.50) | | (3-12) | (1-48)) |
| (% DOWSIL 9041) | | | | |
| Microcristalline wax (obtained | | 0.68 | | |
| from petroleum jelly at the rate | | | | |
| of 3.4%) | | | | |
| Dimethicone from DOWSIL | 3.82 | | 2.65 | 1.26 |
| 9041 | | | | |
| Dimethicone (KF96A-10CS) | 10.00 | | 20.00 | 25.93 |
| Dimethicone (Xiameter | | 12.92 | | |
| PMX-200 fluid 350 cS) | | | | |
| Squalane | 4.50 | | 3.12 | |
| Ethylhexyl polyhydroxystearate | | | | 3.70 |
| (Dub Estoline) | | | | |
| Paraffinum liquidum | | 2.38 | | |
| Dicaprylyl carbonate | | 17 | | |
| PEG-9 polydimethylsiloxyethyl | 2.70 | 0.68 | 2.35 | 1.85 |
| dimethicone (KF6028) | | | | |

The powders constituting the pulverulent phase are premixed.

The fatty binder comprising the fatty phase (liquid and solid) is then prepared.

The powders are mashed in with the fatty binder by extrusion.

The compositions are then shaped by pressing.

The compositions obtained exhibit good cohesion and good impact resistance. In particular, they can be stored and transported without crumbling or cracking. Their texture allows easy pick-up and application.

1.-19. (canceled)

20. A solid cosmetic composition comprising:
12 to 50% by weight of a non-volatile liquid fatty phase, and
40 to 85% by weight of a pulverulent phase comprising spherical fillers surface-treated with a metallic soap,
the percentages being expressed by weight, relative to the total weight of the composition.

21. The solid cosmetic composition according to claim 20, wherein the pulverulent phase further comprises a lamellar filler surface-treated with a metallic soap.

22. The solid cosmetic composition according to claim 20, wherein the solid cosmetic composition comprises:
13 to 35% by weight of non-volatile liquid fatty phase, and
50 to 80% by weight of pulverulent phase, preferably from 60 to 75% by weight.

23. The solid cosmetic composition according to claim 20, wherein the pulverulent phase comprises nacres and/or pigments, optionally surface-treated with a metallic soap, and preferably at least nacres, optionally surface-treated with a metallic soap.

24. The solid cosmetic composition according to claim 20, wherein the metallic soap is a soap of fatty acids having from 12 to 22 carbon atoms, and in particular from 12 to 18 carbon atoms.

25. The solid cosmetic composition according to claim 20, wherein the metal of the metallic soap is zinc or magnesium.

26. The solid cosmetic composition according to claim 20, wherein the metallic soap is selected from zinc laurate, magnesium stearate, magnesium myristate, zinc stearate, and mixtures thereof, and preferably the metallic soap is magnesium stearate.

27. The solid cosmetic composition according to claim 20, wherein the pulverulent phase comprises lamellar and spherical fillers in a lamellar/spherical ratio ranging from 1/10 to 10/1, preferably from 1/5 to 9/1.

28. The solid cosmetic composition according to claim 20, wherein the solid cosmetic composition comprises from 0.1 to 15% by weight, preferably from 0.5 to 10% by weight, of a solid fatty phase.

29. The solid cosmetic composition according to claim 20, wherein the non-volatile liquid fatty phase comprises at least one non-volatile oil selected from hydrocarbonated oils, non-phenylated siliconated oils, and mixtures thereof.

30. The solid cosmetic composition according to claim 29, wherein the non-volatile oil is selected from hydrocarbonated oils, and comprising from 30% to 50% by weight, relative to the total weight of the composition, of nacres surface-treated by metallic soap.

31. The solid cosmetic composition according to claim 29, wherein the non-volatile oil is selected from non-phenylated siliconated oils.

32. The solid cosmetic composition according to claim 20, wherein the solid cosmetic composition is free of volatile liquid fatty phase.

33. The solid cosmetic composition according to claim 20, wherein the solid cosmetic composition is free of water.

34. The solid cosmetic composition according to claim 20, further comprising a film-forming polymer.

35. The solid cosmetic composition according to claim 20, further comprising a silicone elastomer.

36. The solid cosmetic composition according to claim 20, wherein the solid cosmetic composition has a hardness of between 30 g and 250 g, preferably between 50 g and 230 g, more preferably between 80 g and 200 g.

37. A process for preparing a solid cosmetic composition according to claim 20, comprising:
premixing the powders constituting the pulverulent phase
preparing a fatty binder comprising the non-volatile liquid fatty phase
mashing the powders in with the fatty binder by extrusion, and
shaping the composition by pressing.

38. A method for making up the skin or lips, consisting of applying to the skin or lips a solid cosmetic composition according to claim 20.
